(19) United States
(12) Patent Application Publication
(10) Pub. No.: US 2018/0229016 A1
(43) Pub. Date: Aug. 16, 2018

(54) VAGINAL APPLICATOR FOR PHARMACEUTICAL PREPARATIONS

(71) Applicant: JKS, LLC, Silver Spring, MD (US)

(72) Inventors: Tracy D. NELSON, Myrttle Beach, SC (US); Francesca M. COOK, Silver Spring, MD (US)

(21) Appl. No.: 15/752,723

(22) PCT Filed: Aug. 25, 2016
(86) PCT No.: PCT/US16/48651

§ 371 Date: Feb. 14, 2018

Related U.S. Application Data
(60) Provisional application No. 62/210,699, filed on Aug. 27, 2015.

(51) Int. Cl. A61M 31/00

(52) U.S. Cl. CPC A61M 31/007; A61M 2210/1475; A61M 2205/581

(57) ABSTRACT

One aspect of the invention provides a vaginal applicator for any solid dosage form of a pharmaceutical preparation. The applicator can be used to transvaginally deliver medicine to the apex of the vaginal canal. Medical conditions that may benefit from transvaginal delivery of medicine include but are not limited to those relating to vaginal diseases as well as conditions where oral ingestion of medications is not feasible or advisable due to gastrointestinal or other issues. The applicator can include three primary components: an applicator barrel, a tip to hold the medication to be delivered, and a plunger. In use, a medical preparation can be placed into the tip. The tip, which conforms to the shape of the medical intervention, firmly grips or envelops the medication depending upon the size. The applicator is then inserted into the vagina and, once in place, the plunger is depressed.

### CROSS REFERENCE TO RELATED APPLICATIONS

This application claims priority to U.S. application 62/210,699, filed on Aug. 27, 2015, the contents of which are incorporated in its entirety.

### FIELD OF THE INVENTION

The present invention generally relates to a device for the transvaginal application of microarrays (e.g., microarray patches), creams, ampules, pills, capsules, ovules, troche, suppositories, rings, or any other solid pharmaceutical formulations for medical, health or other related purposes.

### BACKGROUND OF THE INVENTION

The vagina allows for the delivery of medicine both locally and systemically to combat a variety of medical conditions. Transvaginal application of medicine is most commonly used in the area of women's health and for conditions for which transvaginal application is preferred or where oral or other routes of administration are not feasible. Vaginal applicators are used to facilitate placement of the medicine, improve ease of use, reduce messiness and increase compliance. Currently, the most commonly used applicators are pre-filled and used to administer contraceptive gels, vaginal moisturizers and anti-fungal creams. These applicators traditionally have been designed to administer liquids, creams and gels and are generally commercially available and packaged with the related medication and/or health aide.

Presently there is no single applicator that can accommodate a variety of forms and sizes of pharmaceutical products. The majority of available applicators dispense gels and creams, the use therefore coats the vaginal walls rather than targeting delivery to the apex of the vagina. Delivery to the apex of the vagina is the optimal location for solid drug uptake.

One exception is VAGIFEM® (estradiol vaginal tablets) by Novo Nordisk A/S of Bagsvaerd, Denmark, which includes a proprietary applicator designed to specifically fit the estradiol tablet. However, there are a number of medications that are used in women's health and for other conditions, for which no uniform easy to use solid dosage applicator exists. The only known related applicator is manufactured in Europe, requires a special configuration to match the shape and size of the medication to be used and has ‘wings’ that must be depressed to dispense the medication. Use of such a device can be difficult for pregnant women, individuals of limited mobility, the overweight and the elderly. Solid medications can be inserted manually, but with manual insertion it is difficult to place the medication at the apex of the vagina and special care must be taken to ensure good hygiene and to avoid pain or injury.

What is needed is an apparatus for the transvaginal application of ampules, pills, capsules, ovules, troche, suppositories, microarrays or any other solid pharmaceutical formulations, and creams for medical, health or other related purposes.

### SUMMARY OF THE INVENTION

The following presents a simplified summary in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

The present invention provides a device for the transvaginal application of ampules, pills, capsules, ovules, troche, suppositories or any other solid pharmaceutical formulations, and creams for medical, health or other related purposes.

It is an object of the invention to facilitate the easy transvaginal delivery of solid dosage pharmaceutical preparation/medicine to the vagina to both improve convenience and compliance.

It is a further object of the invention to provide a device that can deliver solid dosage forms in a variety of sizes and shapes

It is an object of the invention to provide a device that can deliver vaginal creams.

It is an object of the invention to provide a device that can deliver microarray technology.

It is a further object of the invention to provide a device that can target delivery of solid dosage forms to the apex of the vagina.

It is a further object of the invention to provide a device having a tip that allows delivery of solid medications ranging from 5 to 15 mm in size.

It is a further object of the invention to provide a device comprising material suitable for use in septic systems.

It is a further object of the invention to provide a device comprising materials that are biodegradable.

It is a further object of the invention to provide a device that does not comprise ‘wings’ that must be depressed to dispense the medication

### BRIEF DESCRIPTION OF THE DRAWINGS

For a more complete understanding of the nature and desired objects of the present invention, reference is made to the following detailed description taken in conjunction with the accompanying drawing figures wherein like reference characters denote corresponding parts throughout the several views.

FIGS. 1A-1F illustrate an applicator of the present invention.

FIG. 2 illustrates a tip of the present invention.

FIG. 3 illustrates a plunger of the present invention.

FIG. 4 illustrates a non-slip grip of the present invention.

FIG. 5 illustrates close-up views of applicator top and applicator bottom of the present invention.

### DETAILED DESCRIPTION

Described herein is a vaginal applicator for solid dosage forms of pharmaceutical preparations, creams, and microarrays. The applicator can be used to transvaginally deliver medicine to the apex of the vaginal canal. The apex of the vaginal canal includes a rich collection of blood vessels enabling the optimal absorption for both local and systemic applications of medical products. Medical conditions that may benefit from transvaginal delivery of medicine include but are not limited to those relating to vaginal diseases and sexually transmitted diseases as well as conditions where oral ingestion of medications is not feasible or advisable due to gastrointestinal or other issues. The applicator can be similar in design to a tampon or syringe applicator. In at least one embodiment the applicator is composed of three parts: an applicator barrel, a specially designed tip to hold the medication to be delivered, and a plunger. The applicator can accommodate a variety of creams, microarrays, and solid medication sizes and shapes. In one embodiment the applicator provides an audible click once the medication is dispensed to provide assurance that the medication has been delivered correctly at the apex of the vagina prior to removing the device.

In at least one embodiment, the applicator is made of a biodegradable material and/or an environmentally friendly material. In at least one embodiment, the applicator is made of a material suitable for use in septic systems. In at least one embodiment, the applicator is made of a material that includes a petroleum-based plastic. In at least one embodiment, the applicator is made of a material that includes an ethanol-based plastic. In at least one embodiment, the applicator material is amino-acid-based. In at least one embodiment, the applicator material is paper- or plant-based.

In one example of use, a medical preparation is placed into the tip. The tip can conform to the shape of the medical preparation and firmly grip or envelop the medical preparation depending upon the size. The applicator can then be inserted into the vagina and, once in place, the plunger is depressed. An audible click can be heard when the medicine is dispensed. The length and design of the device can ensure optimal placement at the apex of the vaginal canal maximizing drug effectiveness. The applicator of the present invention provides a safe, easy and hygienic alternative to manual placement.

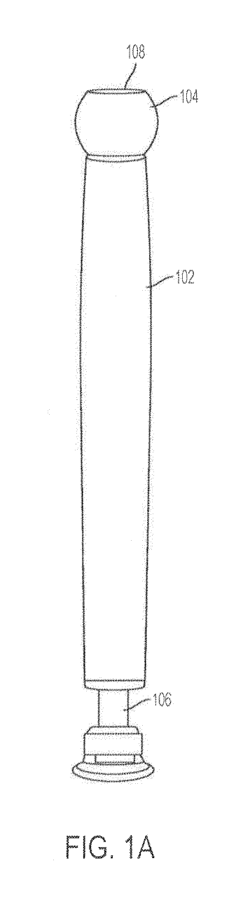

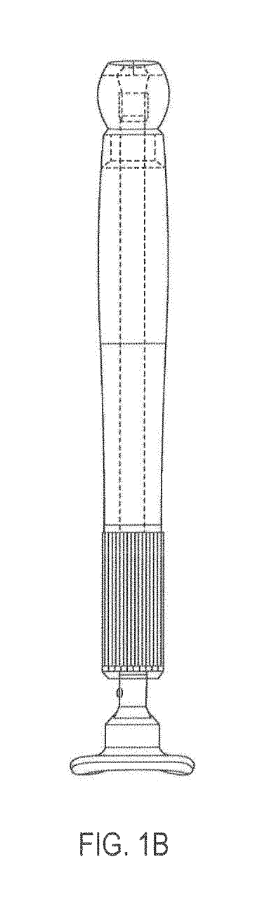

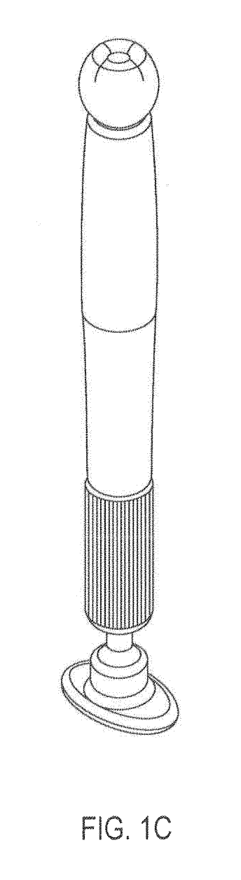

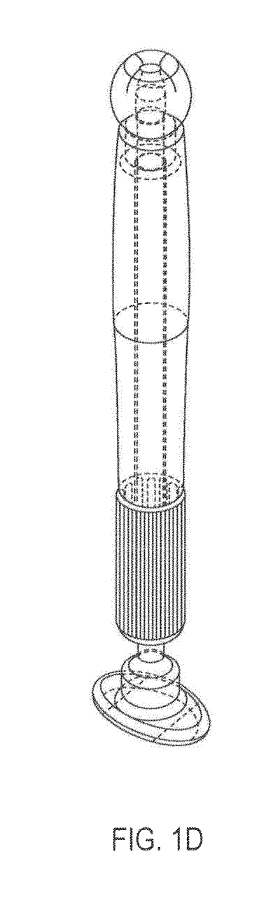

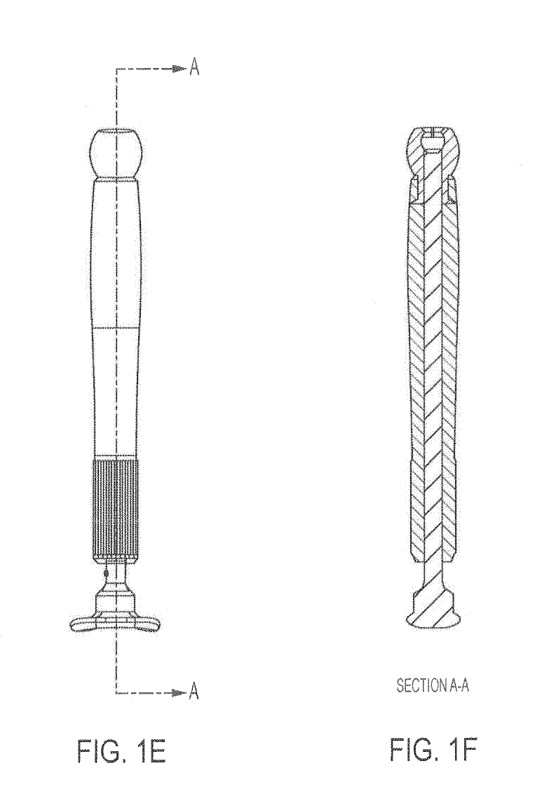

As shown in FIGS. 1A-1E the applicator can include an outer hollow barrel 102 with tip 104 and inner plunger 106. Outer barrel 102 can be tubular in shape. In one embodiment tip 104 is attached to and covers the top of outer barrel 102. In another embodiment tip 104 is attachable to the outer barrel 102 and when attached covers the top of outer barrel 102. Plunger 106 can be circular in cross-section and fit inside of outer barrel 102. The top of plunger 106 can be circular in cross-section and flat and matches the diameter of opening 108. When plunger 106 is depressed, it can fully push into tip 104 ejecting its contents. Outer barrel 102 can be a hollow tube allowing plunger 106 to move freely. In at least one embodiment, the applicator can be designed as one piece. In at least one embodiment, the applicator can be made from plant-based material and biodegradable. In at least one embodiment the applicator is reusable (e.g., allowing for at least five or ten uses). In at least one embodiment the applicator comprises biodegradable starch-based plastic and silicon. In at least one embodiment outer barrel 102 is 127 to 152 mm in length. In at least one embodiment, at the time of administration, outer barrel 102 is fully inserted approximately 76 to 102 mm into the vaginal canal ensuring delivery into the apex of the vagina.

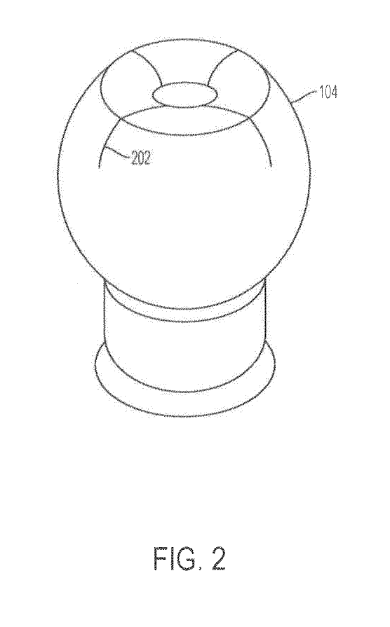

As shown in FIG. 2, the applicator can include tip 104. Tip 104 can provide for a firm grip of the medicament regardless of the size or shape of the medical product being inserted. Tip 104 can hold solid pharmaceutical preparations that range from approximately 0.5 mm to 15 mm in width. In at least one embodiment, tip 104 is soft, malleable and/or rounded to ensure comfortable insertion. For example, the tip 104 can have curves with radii between about 2 mm and about 8 mm. In one embodiment, the radius of the tip 104 when viewed from the side as depicted in FIGS. 1A, 1B, 1E, 1F, and 5 can be between about 2 mm and about 3 mm. In another embodiment, the radius of the tip when viewed from the top or bottom can be between about 7 mm and about 8 mm.

Tip 104 can be attached to the proximal end of outer barrel 102, covering the entire opening of outer barrel 102. Tip 104 can serve as a medication chamber. In one embodiment tip 104 is made from a material-having memory. That is, tip 104 maintains and/or returns to its shape after insertion. In at least one embodiment, tip 104 is concave and shaped similar to an ear bud or a blossom. In at least one embodiment tip 104 is approximately 13 mm to 15 mm in length. In at least one embodiment the diameter of tip 104 opening is approximately 5 mm. In at least one embodiment tip 104 can firmly grip medications larger than 10 mm. Smaller medications can be fully encased inside tip 104 ensuring that the medication is held in place until dispensed. In at least one embodiment, tip 104 further includes cut(s) or slit(s) 202 that allow the tip to spread open like a blossom, which is particularly advantageous for dispensing of larger diameter medications. In at least one embodiment, tip 104 includes at least four cut(s) 202. In at least one embodiment, cuts 202 are extruded cuts. In use, cut(s) 202 allow tip 104 to open as a solid pharmaceutical preparation is pushed through tip 104.

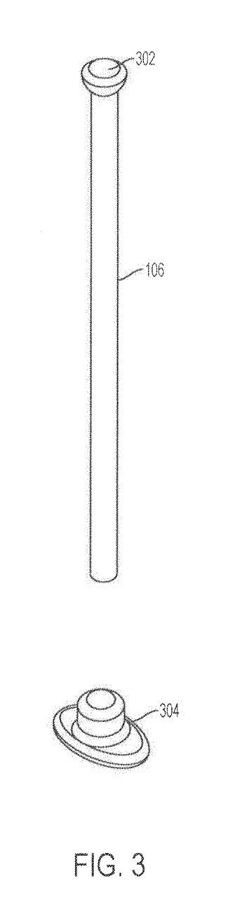

As shown in FIG. 3, plunger 106 is narrower than outer barrel 102 and round. The distal end or plunger top 302 can be flat and circular and covers the width of tip 104. In one embodiment, the outer end of the plunger is ergonomically designed to curve, thereby making depressing the plunger easy and comfortable. When the plunger is depressed, plunger top 302 traverses a partial length of the barrel 102 (e.g., approximately 30-40 mm) and about 10 mm into tip 104, thereby ejecting the enclosed solid pharmaceutical preparation into the apex of the vagina. In at least one embodiment, plunger is attachable to plunger base 304. In at least one embodiment, plunger 106 and plunger base 304 are one unit.

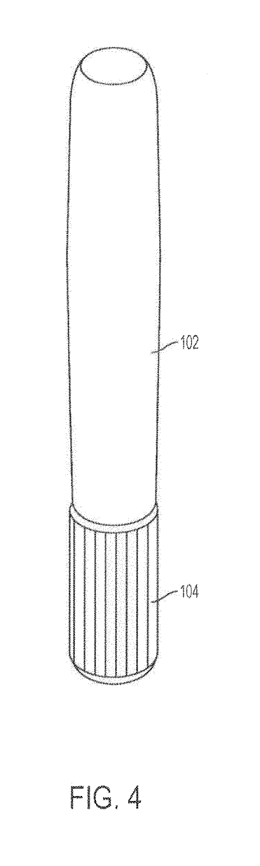

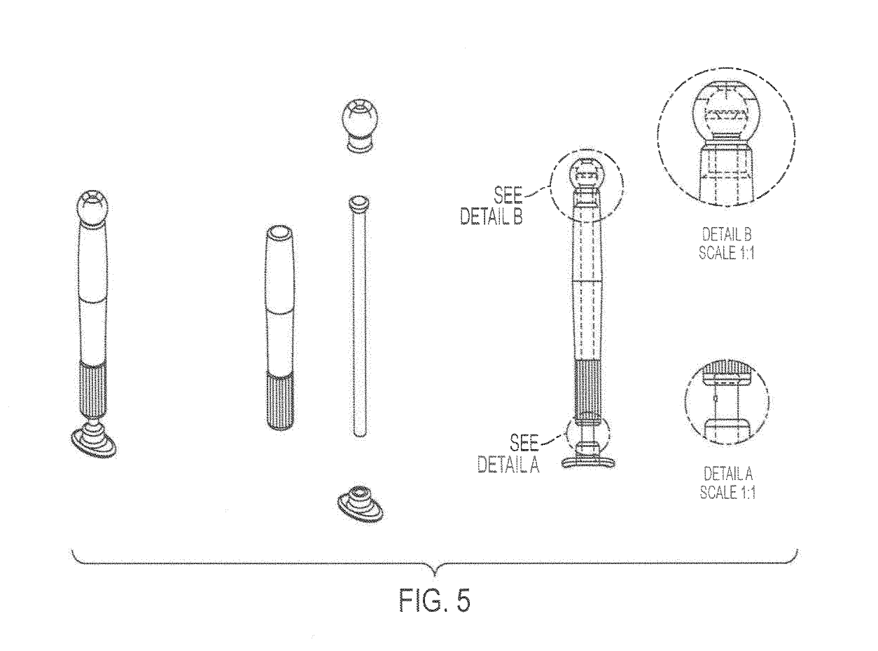

As shown in FIG. 4, in at least one embodiment, outer barrel 102 provides non-slip grip 402. In at least one embodiment, this non-slip grip is provided by a plurality of raised vertical bands at the bottom end of outer barrel 102. In one embodiment, three bands are provided. In an alternative embodiment, a plurality of bands are provided. In a further alternative embodiment, horizontal (i.e., circumferential) bands are provided as opposed to vertical bands that run substantially parallel to a central access of outer barrel 102. Non-slip grip 402 can aid applicator insertion, particularly when lubricants are used.

FIG. 5 provides close-up views of applicator top 502 and applicator bottom 504. In the close-up view of applicator top 502, outer barrel 102, tip 104, opening 108, cuts 202 and plunger top 302 are shown. In the close-up view of applicator bottom 504, non-slip grip 402, plunger 106, plunger base 304, and notification knob 506 are shown. In at least one embodiment, when plunger 106 is depressed and the pill is ejected. notification knob 506 clicks into a circular opening at the bottom of the outer barrel making an audible click and thereby providing notification of pill release. Notification knob 506 can have a rounded shape and extend slightly proud of applicator bottom 504 so that as applicator bottom 504 is advanced, the applicator bottom 504, the outer barrel 102, and/or the notification knob 506 will move across the inside of outer barrel 102 producing a clicking sound.

There are several oral medications that are commonly administered vaginally, for example, misoprostol for cervical ripening, bromocriptine for the treatment of hyperprolactinemia, and indomethacin for arthritic conditions. As one illustrative example, progesterone is one of many products that would benefit from a vaginal applicator such as disclosed herein. Progesterone is generically available. One use of progesterone is to treat premature shortening of the cervix during pregnancy. The availability of an applicator that can accommodate any dosage of progesterone would facilitate self-administration, thereby reducing the need for costly medical assisted methods and likely improve compliance.

As an illustrative summary and as described above, the applicator can include an outer applicator barrel, a plunger, and a tip. The applicator barrel can be made from plant-based material and the tip can be made from a biomedical and/or biocompatible polymer. The applicator can be designed as one piece and can be fully biodegradable. In use, a medical preparation can be placed into the tip. The tip can firmly grip and/or envelop the medication, depending on size, to be delivered transvaginally. The applicator can be placed in the vaginal canal and, once in place, the plunger can be depressed. An audible click can be heard when the medicine has been dispensed.

The present invention provides multiple advantages to that which is presently available. These advantages include but are not limited to: ensuring of optimal placement of the medication at the apex of the vagina, accommodating all types of solid preparations including ampules, pills, capsules, ovules, troche, suppositories or any other solid pharmaceutical formulations as well as microarrays for medical or health related purposes, accommodating all sizes and shapes of medication from approximately 5 to 15 mm in width, a similar design to a tampon or syringe applicator that women are familiar with and comfortable using, a tip that grips large solid preparations (e.g., pills) and encases smaller ones to ensuring accurate dispensing, accommodation of vaginal creams, an ergonomic bottom of the plunger that curves to make depressing the plunger easy and comfortable, a soft and malleable tip for providing a comfortable insertion into the vagina, an audible click that informs the user that the medication has been dispensed, a non-slip grip to ensure proper insertion and placement as well as ease of use if using a lubricant, improved hygiene relative to manual insertion that reduces the likelihood of injury or pain, and patient convenience that is likely to improve compliance.

It is thought that the applicator of the present invention will be understood from the foregoing description and it will be apparent that various changes may be made in the form, or manufacture thereof without departing from the spirit and scope of the invention or sacrificing all of its material advantages, the form hereinbefore described being merely a preferred or exemplary embodiment thereof.

What is claimed is:

1. An applicator comprising:
a barrel, wherein said barrel is tubular;
a tip, wherein said tip is attached to, attachable to, or connected with said barrel, said tip comprising a plurality of cuts, wherein said cuts allow for the insertion and/or expulsion of a medicament; and
a plunger, wherein said plunger is circular in cross-section and wherein the cross section of said plunger fits inside said barrel.

2. The applicator of claim 1, wherein said applicator is biodegradable.

3. The applicator of claim 1, wherein said medicament is a solid medicament.

4. The applicator of claim 3, wherein said solid medicament has a width of about 5 mm inches to about 15 mm.

5. The applicator of claim 1, wherein insertion of said plunger causes expulsion of said medicament through said plurality of cuts.

6. The applicator of claim 1, wherein said tip is at least one of soft, malleable, and rounded.

7. The applicator of claim 6, wherein said tip has a hardness of between about 40 Shore A and about 60 Shore A.

8. The applicator of claim 6, Wherein said tip has a hardness of about 50 Shore A.

9. The applicator of claim 6, wherein said tip has an elongation at break between about 90% and about 110%.

10. The applicator of claim 6, wherein said tip has an elongation at break of about 100%.

11. The applicator of claim 6, wherein said tip has a height between about 10 mm and about 25 mm.

12. The applicator of claim 1, wherein said tip is about 5 mm to about 15 mm in length.

13. The applicator of claim 1, wherein said applicator does not comprise wings.

14. The applicator of claim 1, further comprising:
a plunger base, wherein said plunger base is attached, attachable to or connected with said plunger; and
a notification knob, wherein said notification knob in located on said plunger, wherein insertion of said plunger into said barrel and said tip causes expulsion of said medicament through said plurality of cuts, and causes insertion of said notification knob into said barrel thereby providing a click sound.

15. The applicator of claim 1, said barrel comprising an outer portion, said outer portion further comprising a plurality of raised vertical bands at a bottom end.

16. The applicator of claim 1, said barrel comprising an outer portion, said outer portion further comprising a plurality of raised horizontal bands at a bottom end.
